(19) United States
(12) Patent Application Publication
(10) Pub. No.: US 2023/0036033 A1
(43) Pub. Date: Feb. 2, 2023

(54) BIPOLAR ENERGY-BASED SURGICAL INSTRUMENTS

(71) Applicant: Covidien LP, Mansfield, MA (US)

(72) Inventors: Saumya Banerjee, Southington, CT (US); Jacob C. Baril, Norwalk, CT (US); Garrett P. Ebersole, Hamden, CT (US); Justin J. Thomas, New Haven, CT (US); Roy J. Pilletere, Middletown, CT (US); Matthew A. Dinino, Newington, CT (US)

(21) Appl. No.: 17/875,753

(22) Filed: Jul. 28, 2022

Related U.S. Application Data
(60) Provisional application No. 63/228,183, filed on Aug. 2, 2021.

(51) Int. Cl. A61B 18/14

(52) U.S. Cl. CPC A61B 18/14; A61B 2018/1475; A61B 2218/002; A61B 2218/007; A61B 2018/1495; A61B 2018/00601

(57) ABSTRACT

An electrosurgical instrument has a hollow shaft portion defining a plurality of fluid ports configured for suction and/or irrigation. The electrosurgical instrument also includes a plurality of different electrode tips configured to detachably couple to the shaft portion. Each of the electrode tips have an active electrode and a return electrode to provide bipolar electrosurgical energy therebetween.

### CROSS-REFERENCE TO RELATED APPLICATION

This application claims the benefit of and priority to U.S. Provisional Patent Application No. 63/228,183, filed Aug. 2, 2021, the entire contents of which is incorporated by reference herein.

### FIELD

The present disclosure relates to surgical instruments and, more specifically, to bipolar energy-based surgical instruments.

### BACKGROUND

In minimally-invasive surgical procedures, operations are carried out within an internal body cavity through small entrance openings in the body. The entrance openings may be natural passageways of the body or may be surgically created, for example, by making a small incision into which a cannula is inserted.

Multi-function surgical instruments are beneficial in that they allow multiple surgical tasks to be performed with a single instrument, obviating the need to alternatingly remove and insert different instruments into the surgical site to perform a surgical task and/or obviating the need for simultaneously inserting multiple instruments into the surgical site to perform a surgical task. Some of these multi-function surgical instruments allow for cauterization, irrigation, suction, maintaining a pneumoperitoneum, and allow for visual inspection of a patient's anatomy.

### SUMMARY

As used herein, the term “distal” refers to the portion that is described which is further from a user, while the term “proximal” refers to the portion that is being described which is closer to a user. Further, any or all of the aspects described herein, to the extent consistent, may be used in conjunction with any or all of the other aspects described herein.

Provided in accordance with aspects of the present disclosure is a bipolar electrosurgical instrument having a plurality of interchangeable, alternatively designed, bipolar energy-enabled distal tips to allow for various use scenarios such as, for example, tissue cutting, tissue coagulation, tissue cutting and coagulation, etc. Utilizing bipolar energy reduces the risk of patient burns, errant current leaks, and capacitive coupling. Bipolar energy also lessens eschar build-up and offers more controlled tissue site impact.

In an aspect of the present disclosure, an electrosurgical instrument is provided that includes a shaft and a plurality of electrode tips. The shaft defines a channel therethrough and has a distal end portion defining a plurality of suction ports. The distal end portion of the shaft defines an open distal end configured to provide suction and/or irrigation therethrough. The electrode tips are configured for detachable receipt in the distal end portion of the shaft. Each of the electrode tips has an active electrode and a return electrode such that each of the electrode tips is configured for conducting bipolar electrosurgical energy between the active and return electrodes thereof.

In aspects, the active and return electrodes of a first of the electrode tips may be configured as jaw members, the active and return electrodes of a second of the electrode tips may be configured as spatulas, and the active and return electrodes of a third of the electrode tips may be configured as elongate probes.

In aspects, a first of the electrode tips may be configured to move relative to the shaft between a retracted position and an extended position.

In aspects, the first electrode tip may be concealed within the distal end portion of the shaft when in the retracted position and may protrude distally from the open distal end of the shaft when in the extended position.

In aspects, each of the electrode tips may be configured to detachably couple to a source of bipolar electrosurgical energy.

In aspects, the electrosurgical instrument may further include a housing having a fluid port configured to connect to a source of irrigation fluid, and a vacuum port configured to connect to a vacuum source.

In aspects, the electrosurgical instrument may further include a first actuator coupled to the housing and configured to selectively couple to each of the electrode tips for deploying and retracting the electrode tips relative to the shaft.

In aspects, the electrosurgical instrument may further include a second actuator coupled to the housing and configured to selectively couple to each of the electrode tips for selectively conducting electrosurgical energy to the active electrode.

### BRIEF DESCRIPTION OF THE DRAWINGS

The above and other aspects and features of the present disclosure will become more apparent in light of the following detailed description when taken in conjunction with the accompanying drawings wherein like reference numerals identify similar or identical elements.

FIG. 1 is a perspective view illustrating an electrosurgical instrument in accordance with an aspect of the present disclosure;

FIG. 2 is a perspective view illustrating a distal end portion of a shaft of the electrosurgical instrument of FIG. 1;

FIG. 3 is a side view illustrating a first electrode tip for coupling to the distal end portion of the shaft of FIG. 2;

FIG. 4 is a side view illustrating a second electrode tip for coupling to the distal end portion of the shaft of FIG. 2; and

FIG. 5 is a side view illustrating a third electrode tip for coupling to the distal end portion of the shaft of FIG. 2.

### DETAILED DESCRIPTION

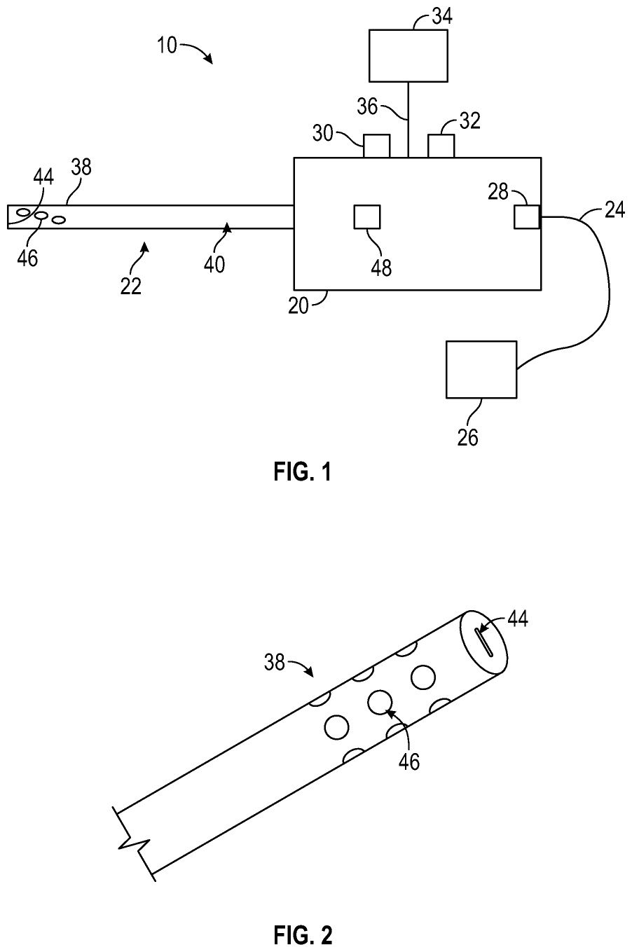

Referring generally to FIGS. 1-5, an electrosurgical instrument is provided that is equipped with irrigation and aspiration functionalities and has a plurality electrode tips each configured for grasping tissue, treating grasped tissue with bipolar energy, and/or mechanically dissecting grasped tissue. Each of the electrode tips is configured to be detachably coupled to a shaft of the electrosurgical instrument and selected depending on their suitability for the intended use.

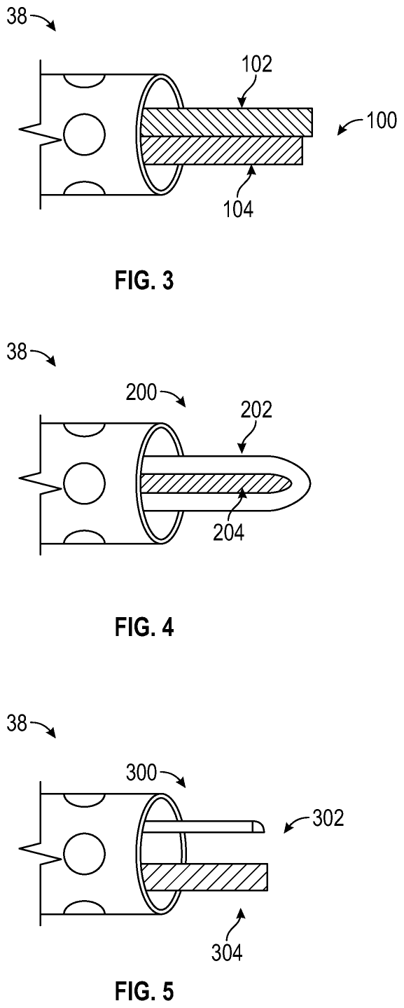

With reference to FIG. 1, an electrosurgical instrument provided in accordance with the present disclosure is shown generally identified by reference numeral 10. Instrument 10 includes a housing 20, an elongated shaft 22, and a plurality of discrete end effectors or electrode tips 100, 200, 300 (FIGS. 3-5) configured to be selectively received within the shaft 22. The instrument 10 also includes an electrosurgical cable 24 that connects instrument 10 to a power source 26, which may be an electrosurgical generator. The electrosurgical cable 24 includes wires (not shown) extending therethrough that have sufficient length to extend through housing 20 and/or shaft 22 in order to provide energy to at least one of the electrode tips 100, 200, 300, e.g., upon activation of a first actuator 28.

The housing 20 has a first button 30, such as, for example, a fluid port and a second button 32, such as, for example, a vacuum port each disposed on housing 20 and which enable connection of instrument 10 to a suction and irrigation source 34 via suitable tubing 36 (integral or removable tubing). Within housing 20 an internal fluid line (not explicitly shown) connects fluid and vacuum ports 30, 32 with the source 34 to enable the delivery of fluid (e.g., water or saline) to and/or withdrawal of fluid from the shaft 22. More specifically, suction and/or irrigation source 34 may be configured to only provide suction or irrigation through shaft 22. Alternatively, suction and/or irrigation source 34 may be configured to provide, in a first configuration, suction though shaft 22, and, in a second configuration, irrigation though shaft 22. In aspects, the buttons 30, 32 may be remote from instrument 10, e.g., on the suction and/or irrigation source 34.

With reference to FIGS. 1 and 2, shaft 22 extends distally from housing 20 and supports a selected one of the plurality of electrode tips 100, 200, 300 at a distal end portion 38 of the shaft 22. Shaft 22 defines a channel 40 therethrough configured for receipt of one of the plurality of electrode tips 100, 200, 300 and for the passage of fluid (e.g., during irrigation) and/or air (e.g., during aspiration). The distal end portion 38 of shaft 22 has an open distal end 44 and defines a plurality of fluid ports or apertures 46 configured for supplying irrigation fluid therefrom and for applying suction to a surgical site. In aspects, the shaft 22 may be rigid, flexible, or configured to be selectively articulated between a plurality of angular orientations.

With reference to FIG. 3, the first electrode tip 100 is configured to be removably received within the channel 40 of shaft 22 and detachably coupled to power source 26 (FIG. 1). The first electrode tip 100 may be detachably operably coupled to a second actuator or trigger 48 (FIG. 1) disposed on housing 20. The trigger 48 is configured to translate the first electrode tip 100 between a retracted position, in which the electrode tip 100 is concealed within the distal end portion 38 of the shaft 22 (FIG. 2), and an extended position (FIG. 3), in which the electrode tip 100 protrudes distally from the open distal end 44 of shaft 22.

The first electrode tip 100 includes a first jaw member 102, which may be an active electrode, and a second jaw member 104, which may be a return electrode. The first and second jaw members 102, 104 may be configured as opposing jaw members pivotably coupled to one another. Each of the jaw members 102, 104 includes an electrically-conductive surface (not explicitly shown) adapted to connect to the power source 26 and defines a bipolar configuration in use wherein the surface of the first jaw member 102 is charged to a first electrical potential and the surface of the second jaw member 104 is charged to a second, different electrical potential such that an electrical potential gradient is created for conducting energy between the surface and through tissue grasped therebetween for treating tissue. The first actuator 28 of the housing 20 is operably coupled between the power source 26 and the surfaces of the jaw members 102, 104 via the electrosurgical able 24, thus allowing the surgeon to apply energy, e.g., bipolar electrosurgical energy, to the surfaces of jaw members 102, 104. In aspects, a knife (not shown) may be movably received between the jaw members 102, 104 of the electrode tip 100 for selectively cutting tissue disposed therebetween.

Referring to FIG. 4, another electrode tip 200 for coupling to the distal end portion 38 of shaft 22 of electrosurgical instrument 10 is shown. Electrode tip 200 includes an active electrode 202 and a return electrode 204 each configured as opposing spatulas.

Referring to FIG. 5, another electrode tip 300 for coupling to the distal end portion 38 of shaft 22 of electrosurgical instrument 10 is shown. Electrode tip 300 includes an active electrode 302 and a return electrode 304 each configured as opposing elongate probes configured to clamp and coagulate tissue disposed therebetween.

It is contemplated that electrosurgical instrument 10 may have one or more other alternatively configured electrode tips, such as, for example, L-shaped, hook-shaped, V-shaped, or the like.

Turning back to FIG. 1, as opposed to handheld, manual manipulation and operation, the various embodiments disclosed herein may also be configured to work with robotic surgical systems and what is commonly referred to as “telesurgery.” Such systems employ various robotic elements to assist the surgeon and allow remote operation (or partial remote operation) of surgical instrumentation. Various robotic arms, gears, cams, pulleys, electric and mechanical motors, etc. may be employed for this purpose and may be designed with a robotic surgical system to assist the surgeon during the course of an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of surgeons or nurses may prep the patient for surgery and configure the robotic surgical system with one or more of the instruments disclosed herein while another surgeon (or group of surgeons) remotely control the instruments via the robotic surgical system. As can be appreciated, a highly skilled surgeon may perform multiple operations in multiple locations without leaving his/her remote console which can be both economically advantageous and a benefit to the patient or a series of patients.

The robotic arms of the surgical system are typically coupled to a pair of control handles by a controller. The handles can be moved by the surgeon to produce a corresponding movement of the working ends of any type of surgical instrument (e.g., end effectors, graspers, knifes, scissors, etc.) which may complement the use of one or more of the embodiments described herein. The movement of the control handles may be scaled so that the working ends have a corresponding movement that is different, smaller or larger, than the movement performed by the operating hands of the surgeon. The scale factor or gearing ratio may be adjustable so that the operator can control the resolution of the working ends of the surgical instrument(s).

The control handles may include various sensors to provide feedback to the surgeon relating to various tissue parameters or conditions, e.g., tissue resistance due to manipulation, cutting or otherwise treating, pressure by the instrument onto the tissue, tissue temperature, tissue impedance, etc. As can be appreciated, such sensors provide the surgeon with enhanced tactile feedback simulating actual operating conditions. The control handles may also include a variety of different actuators for delicate tissue manipulation or treatment further enhancing the surgeon's ability to mimic actual operating conditions.

From the foregoing and with reference to the various drawings, those skilled in the art will appreciate that certain modifications can be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. An electrosurgical instrument, comprising:
a shaft defining a channel therethrough and having a distal end portion defining a plurality of suction ports, the distal end portion of the shaft defining an open distal end configured to provide at least one of suction or irrigation therethrough; and
a plurality of electrode tips configured for detachable receipt in the distal end portion of the shaft, wherein each of the plurality of electrode tips has an active electrode and a return electrode such that each of the plurality of electrode tips are configured for conducting bipolar electrosurgical energy between the active and return electrodes.

2. The electrosurgical instrument according to claim 1, wherein the active and return electrodes of a first electrode tip of the plurality of electrode tips are configured as jaw members, the active and return electrodes of a second electrode tip of the plurality of electrode tips are configured as spatulas, and the active and return electrodes of a third electrode tip of the plurality of electrode tips are configured as elongate probes.

3. The electrosurgical instrument according to claim 1, wherein at least a first electrode tip of the plurality of electrode tips is configured to move relative to the shaft between a retracted position and an extended position.

4. The electrosurgical instrument according to claim 3, wherein the first electrode tip is concealed within the distal end portion of the shaft when the first electrode tip is in the retracted position, and the first electrode tip protrudes distally from the open distal end of the shaft when the first electrode tip is in the extended position.

5. The electrosurgical instrument according to claim 1, wherein each of the plurality of electrode tips is configured to detachably couple to a source of bipolar electrosurgical energy.

6. The electrosurgical instrument according to claim 1, further comprising a housing having a fluid port configured to connect to a source of irrigation fluid, and a vacuum port configured to connect to a vacuum source.

7. The electrosurgical instrument according to claim 6, further comprising a first actuator coupled to the housing and configured to selectively couple to each of the plurality of electrode tips for deploying and retracting the plurality of electrode tips relative to the shaft.

8. The electrosurgical instrument according to claim 7, further comprising a second actuator coupled to the housing and configured to selectively couple to each of the plurality of electrode tips for selectively conducting electrosurgical energy to the active electrode.
